(19) United States
(12) Patent Application Publication
(10) Pub. No.: US 2016/0282359 A1
(43) Pub. Date: Sep. 29, 2016

(54) MICROFABRICATED QLIDA BIOSENSORS WITH AN EMBEDDED HEATING AND MIXING ELEMENT

(71) Applicants: Elisabeth Papazoglou, Yardley, PA (US); Hongseok Noh, Ambler, PA (US); Chengjie Yu, Philadelphia, PA (US); Peter M. Clark, Philadelphia, PA (US)

(72) Inventors: Elisabeth Papazoglou, Yardley, PA (US); Hongseok Noh, Ambler, PA (US); Chengjie Yu, Philadelphia, PA (US); Peter M. Clark, Philadelphia, PA (US)

(73) Assignee: Drexel University, Philadelphia, PA (US)

(21) Appl. No.: 15/172,096

(22) Filed: Jun. 2, 2016

Related U.S. Application Data
(62) Division of application No. 14/043,532, filed on Oct. 1, 2013, now abandoned.
(60) Provisional application No. 61/708,399, filed on Oct. 1, 2012.

(51) Int. Cl. G01N 33/58; B01L 3/00; G01N 21/64; G01N 33/543; G01N 33/545

(52) U.S. Cl. CPC G01N 33/588; G01N 33/5438; G01N 33/545; G01N 33/54393; G01N 21/6428; B01L 3/50273; B01L 3/502761; G01N 2021/6439; B01L 2400/0418; B01L 2300/0654

(57) ABSTRACT

An apparatus and method for detecting an analyte are described. The apparatus includes at least one microchannel adapted for an analyte to adhere to an interior surface thereof, a mixing element positioned within at least a portion of the at least one microchannel, a light source for energizing quantum dots conjugated with the analyte within the at least one microchannel, and a detection system for detecting and quantifying fluorescent energy emitted by the quantum dots in one or more predetermined wavelength ranges, wherein each wavelength range being correlated to one and only one type of analyte. The method includes the steps of providing a sample to at least one microchannel coated with an antibody, contacting the sample with a conjugate comprising a quantum dot and an antibody that specifically binds to the analyte, increasing electrothrermal flow of the sample, energizing the quantum dot with a light source, detecting fluorescent emission from the quantum dot, and correlating the fluorescent emission to the presence of or the concentration of the analyte in the sample.

### CROSS-REFERENCE TO RELATED APPLICATIONS

This application is a divisional of U.S. patent application Ser. No. 14/043,532, filed on Oct. 1, 2013, which claims priority to U.S. Provisional Application Ser. No. 61/708,399, filed Oct. 1, 2012. The entire content of each disclosure are hereby incorporated by reference herein in their entirety.

### BACKGROUND OF THE INVENTION

Optically transduced microfluidic immunoassays have proven to be a highly sensitive and rapid method to assess the concentrations of analytes within biological samples. The advantages that microfluidic immunosensor platforms have over standard microtiter plate based assays arise from their higher surface area to volume ratio, allowing smaller working volumes, decreased reagent consumption and shorter characteristic diffusion lengths for biomolecules.

Although microfluidic immunoassays facilitate higher throughput and automation than standard microtiter plates, the immunoreaction within such devices remains diffusion limited unless, there is a way to achieve recursive or continuous sample flow. The present invention satisfies this need.

### SUMMARY OF THE INVENTION

The present invention includes an apparatus for detecting an analyte. The apparatus comprises at least one microchannel adapted for an analyte to adhere to an interior surface thereof, a mixing element positioned within at least a portion of the at least one microchannel, a light source for energizing quantum dots conjugated with the analyte within the at least one microchannel, and a detection system for detecting and quantifying fluorescent energy emitted by the quantum dots in one or more predetermined wavelength ranges, where each wavelength range being correlated to one and only one type of analyte.

In one embodiment, the mixing element is a resistive heating element. In one embodiment, the mixing element is a resistive heating element comprises at least one mirco-patterned metal wire. In one embodiment, the mixing element comprises at least one electrode.

In one embodiment, the at least one microchannel comprises a transparent polymer material. In one embodiment, the at least one microchannel comprises polymethyl methacrylate (PMMA), polyvinyl acetate, polycarbonate, or polystyrene. In one embodiment, the microchannel comprises at least one silicon substrate having a conductive layer. In one embodiment, the mixing element comprises at least one electrode patterned from the conductive layer.

In one embodiment, the light source comprises an LED. In one embodiment, the light source further comprises a lens for focusing the LED onto the at least one microchannel.

In one embodiment, the detection system comprises a broadband filter. In one embodiment, the detection system comprises a photodetector. For example, in one embodiment, the photodetector is a spectrometer coupled to at least one photomultiplier tube. In one embodiment, the photodetector is a CCD camera. In one embodiment, the detection system further comprises a fiber optic for transmitting light from the at least one microchannel to the photodetector.

In one embodiment, the apparatus of the invention comprises a composition for detecting an analyte in a biological sample contained in the at least one microchannel, where the composition comprises at least one conjugate comprising a quantum dot and an antibody that specifically binds to the analyte.

The present invention includes a method of detecting an analyte in a sample. The method comprises providing a sample to at least one microchannel coated with an antibody, the sample potentially including an analyte; contacting the sample with a conjugate comprising a quantum dot and an antibody that specifically binds to the analyte; increasing electrothrermal flow of the sample; energizing the quantum dot with a light source; detecting fluorescent emission from the quantum dot; and correlating the fluorescent emission to the presence of or the concentration of the analyte in the sample.

In one embodiment, electrothermal flow is increased by applying a voltage to an electrode within at least a portion of the at least one microchannel. In one embodiment, electrothermal flow is increased by DC electro-osmotic transverse mixing.

In certain embodiments, the analyte is an enzyme, an adhesion molecule, a cytokine, a protein, a lipid mediator, an immune response mediator, or a growth factor.

In one embodiment, the applied voltage is about 1 Vrms to about 10 Vrms. In one embodiment, the voltage is applied at a frequency of about 0.1 Hz to about 100 MHz. In one embodiment, the applied voltage is about 6 Vrms applied at a frequency of about 200 kHz.

In one embodiment, the volume of the sample is about 0.1×10⁻¹⁰ m³ to about 0.1×10⁻⁵ m³. In one embodiment, the volume of the sample is less than or equal to about 1 μL.

In one embodiment, the detection of the analyte occurs in about 1 minute to about 60 minutes.

The present invention includes a device for detecting an analyte. The device comprises at least one rectangular microchannel formed between a polymethyl methacrylate (PMMA) substrate and a silicon substrate, the at least one microchannel having a width of about 220 μm and a height of about 110 μm; an immobilized capture agent positioned on an interior lumen surface of the at least one rectangular microchannel, where the capture agent specifically binds to the analyte; and an electrode patterned from a conductive layer on the silicon substrate, the electrode positioned within at least a portion of the at least one microchannel.

In one embodiment, the electrode is a longitudinal electrode. In one embodiment, the conductive layer comprises a material selected from the group consisting of aluminum and chromium. In one embodiment, the electrode is adapted to receive an applied voltage.

In one embodiment, the immobilized capture agent is an antibody.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts a set of schematics detailing exemplary fabrication protocols of exemplary devices of the present invention. FIG. 1A depicts the fabrication flow for all PMMA microchannels. FIG. 1B depicts the fabrication of PMMA-Si microchannel with an embedded mixing element.

FIG. 2 depicts an exemplary microchannel excitation and imaging setup.

FIG. 3 is a graph depicting the numerical simulation result of fluid velocity as a function of the applied voltage. Frequency was fixed at 200 kHz. At 6 Vrms, the velocity is 296 μm/s.

FIG. 4, comprising FIG. 4A and FIG. 4B, is a set of graphs depicting the comparison of capillary-based and microfabricated QLISA. FIG. 4A is a lactoferrin calibration curve comparison between capillary and rectangular microchannel. FIG. 4B depicts a QLISA assay intensity with and without ETF mixing. Significant increase in the intensity is observed with ETF mixing. The electrode area gives a higher signal than non-electrode area in the case of ETF mixing.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Detection Device and System

The present invention provides a system, apparatus and device for improved immunoassay performance and sensitivity. The device of the invention comprises an immunoassay biosensor platform comprising one or more microchannels having an embedded heating and/or mixing element within the microchannels. This heating element allows for superior mixing of the sample and reagents of the immunoassay, which significantly increases sensitivity of the assay, reduces reagent consumption, and reduces incubation times.

In one embodiment, the invention comprises a microfabricated quantum-dot-linked-immuno-diagnostic-assay (QLIDA) biosensor with an embedded heating element and/or mixing element within at least a portion of a microchannel. As demonstrated herein, the biosensor exhibits comparable or improved sensitivity to ELISA, but requires significantly less sample and reagent volumes (e.g., 1/100 of traditional assays). The heating and mixing within the microchannels of the device enhances biosensor kinetics, resulting in rapid transfer of biomolecules to the sensor surfaces. In certain embodiments, the device and method of the invention is used for the detection of a compound or analyte of interest, including for example a protein, peptide, nucleic acid, biomolecule, biomarker, and the like, in a sample. For example, the present invention provides an improved quantum dot-based assay platform for diagnostic or screening assays.

In one embodiment, the device comprises one or more hermetically sealed microchannels. For example, in one embodiment, the microchannels of the device are formed between two substrates. In certain embodiments, one or more of the substrates is polymethyl methacrylate (PMMA). PMMA is a preferred substrate material because of its optical properties and the capability to selectively functionalize its surface for antibody immobilization.

The substrate of the device is not limited to PMMA. Rather any material, including for example glass and polymers such as polystyrene, polyvinylacetate, polyvinylchloride, polymetheylpentene, and polycarbonate, may be used in the present device. In certain embodiments, a transparent polymer material capable of transmitting light down to about 365 nm can be used as one or more of the substrates of the present device. Polymeric substrates are of particular interest due to readily available functional groups on their surface offering an appropriate substrate for immobilizing antibodies or antigens. Furthermore, photochemical methods are available to functionalize polymeric materials. Several strategies have been devised to detect low concentrations of antigens by solid phase immunoassay in channels, primarily focusing on excitation of fluorophores followed by collection of the emitted photons. One such approach takes advantage of the evanescent field at the interface of the polymer/liquid interface for collecting the emitted signal; this particular method requires substrate material surrounding the channel to function as a waveguide. The excellent optical properties of PMMA allow use of PMMA substrates as waveguides and fabrication of sensors to measure optical properties of molecules.

In one embodiment, the device of the present invention comprises rectangular microchannels. For example, it is described herein that sensors comprising rectangular microchannels exhibit greater assay sensitivity as compared to those comprising cylindrical capillary tubes.

In one embodiment, the rectangular microchannels have a width of about 1 μm to about 1 mm. In one embodiment, the rectangular microchannels have a width of about 100 μm to about 500 μm. In one embodiment, the rectangular microchannels have a width of about 220 μm.

In one embodiment, the rectangular microchannels have a height of about 1 μm to about 1 mm. In one embodiment, the rectangular microchannels have a height of about 50 μm to about 500 μm. In one embodiment, the rectangular microchannels have a height of about 110 μm.

In one embodiment, the rectangular microchannels have a length of about 1 mm to about 100 cm. In one embodiment, the rectangular microchannels have a length of about 1 cm to about 10 cm. In one embodiment, the rectangular microchannels have a length of about 3 cm.

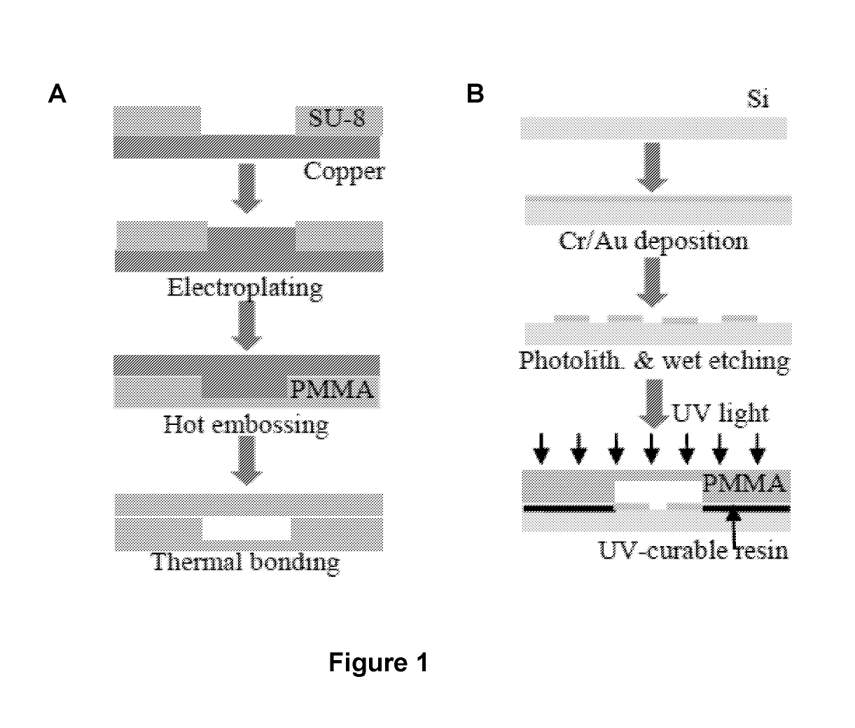

In certain embodiments, the microchannels of the device are formed between two PMMA substrates, as shown in FIG. 1A. In another embodiment, the microchannels of the device are formed between a PMMA substrate and a silicon substrate, whose surface comprises integrated electrodes. Sealing of the PMMA substrate over the silicon substrate thereby produces microchannels with embedded electrodes, as shown in FIG. 1B. The electrodes may be patterned upon the surface of the silicon substrate using any known methodology in the art, including for example photolithography. For example, in certain embodiments, conductive material (e.g., chromium, aluminum, or the like) is deposited upon a silicon substrate. In one embodiment, patterned electrodes are formed via etching of portions of the deposited conductive layer.

In one embodiment, the device comprises embedded electrodes positioned within at least a portion of one or more microchannels of the device, which facilitates electrothermal flow of the sample resulting from joule heating. For example, in one embodiment, the device comprises a resistive heating element integrated into at least a portion of one or more microchannels of the device. In one embodiment, the heating element comprises micro-patterned metal wires. In one embodiment, the device comprises embedded electrodes within at least a portion of one or more microchannels of the device. In certain embodiments, a high frequency voltage is applied to the embedded electrodes, providing enhanced electrothermal flow within at least a portion of the sample. In certain embodiments, the electrodes are longitudinal electrodes, wherein DC electro-osmotic transverse mixing is implemented with DC switching at low frequency voltage (less than a few Hz).

In one embodiment, one or more microchannels comprise a luminal wall that is functionalized with a capture agent, for example a nucleic acid or antibody, which specifically binds to a compound of interest. In certain embodiments, the wall comprises poly(ethylene glycol) (PEG) molecules which crosslink the capture agent to the microchannel wall, as described for example in WO/2011/143456, which is herein incorporated by reference in its entirety. The relative amount or density of the capture agent may be varied as necessary to provide a device suitable for an assay with a desired specificity. In certain embodiments, one or more microchannels may comprise pronounced and/or varied surface features to increase the surface area of the channel walls, thereby providing a larger area for capture agent immobilization. In certain embodiments, the microchannel comprises microspheres functionalized with the capture agent.

In one assay method using the device as disclosed herein, antigen can be captured and analyzed at levels ranging as low as picograms to nanograms. In one embodiment, the method comprises providing a device described herein, coating at least a portion of the microchannel surface with antigen, blocking antigens that are not disease specific, and binding disease specific conjugates to the remaining antigens. In another embodiment, the method comprises functionalizing a microchannel, conjugating an appropriate polyclonal antibody specific to the antigen desired to be measured within the microchannel, reacting the antibody-antigen complex with a quantum dot tagged secondary antibody specific to the antigen, exposing the microchannel to a light source to excite the quantum dots, and determining the concentration of the antigen by measuring the fluorescence of the quantum dots. In particular, an untreated microchannel is coated with antigen, nonspecific antigens are blocked with antibodies such as immunoglobulin, and specific antigens are bound to quantum dot conjugated antibodies.

In certain embodiments, voltage is applied to the embedded electrodes of the microchannel during incubation of a sample comprising a target compound, incubation of the primary antibody, incubation of the quantum dot tagged secondary antibody, or a combination thereof. The applied voltage results in electrothermal flow of the target and/or antibodies in the sample, which increases the velocity of the target and antibodies and improves the performance and sensitivity of the assay. In certain embodiments, the method comprises applying either AC or DC voltage. The amplitude, frequency, and mean (e.g., Vrms), of the applied voltage may be varied depending upon the desired sensitivity, desired speed of the assay, type of assay being performed, and the like. In one embodiment, the applied voltage is about 0.1 Vrms to about 100 Vrms. In one embodiment, the applied voltage is about 1 Vrms to about 10 Vrms. In one embodment, the applied voltage is about 6 Vrms. In one embodiment, the frequency of the applied voltage is about 0.1 Hz to about 100 MHz. In one embodiment, the frequency of the applied voltage is about 1 kHz to about 1 MHz. In one embodiment, the frequency of the applied voltage is about 200 kHz.

As described herein, the device and method of the present invention allows for the use of smaller quantities of samples and reagents as compared to prior biosensor platforms. For example, the electrothermal mixing provided by the embedded mixing element improves sensitivity, allowing for the use of smaller reagent quantities, including smaller volumes of a sample of interest, an antibody comprising solution, quantum-dot-complex comprising solution, or the like. In one embodiment, the method comprises using a sample and/or reagent volume of about 0.1×10⁻¹⁰ m³ to about 0.1×10⁻⁵ m³. In one embodiment, the method comprises using a sample and/or reagent volume of about 7.26×10⁻¹⁰ m³. For example, the present invention allows for the use of sample and/or reagent volumes on the order of nanoliters or microliters, which is substantially smaller than prior platforms. In one embodiment, the sample and/or reagent volume is less than or equal to about 1 μL.

As described herein, the device and method of the present invention allows for reduced assay time. This allows for conducting an assay, using the device and method of the present invention, on the order of minutes. For example, in one embodiment, an assay is conducted in about 1 minute to about 60 minutes. As contemplated herein, excitation photons can be provided directly through one or both ends or through the sidewall of the microchannels, and emitted photons can be collected into a spectrometer or CCD camera either directly or via a fiber optic cable. Alternatively, other optical emission detectors, including but not limited to photomultiplier tubes (PMTS), avalanche photodiode detectors (APDs), and multi-pixel photon counters (MPPCc) can be used.

In another embodiment, the present invention includes a testing apparatus that includes at least one microchannel for containing a sample tagged with quantum dot conjugates, an LED light source to excite the quantum dots, a spherical or flat mirror to concentrate the fluorescence emitted by the quantum dots, and an optical detection system for detecting and measuring the fluorescence signal. Alternatively, a reflector or mirror can be used to concentrate the fluorescent emissions from the quantum dots. The present invention also includes use of an optical detection system that may include a broadband filter to improve signal quality, and can further utilize a photodiode based detector, a spectrometer with one or more photomultiplier tubes, a CCD camera, or other optical detection system, depending on the sensitivity required. In one embodiment, quantum dot intensity may be measured using a standard fluorescence meter (Fluoromax 3), which permits determination of quantum dot bioconjugate concentrations.

In one embodiment, the system comprises a power source for delivering an applied voltage to the embedded mixing element of the microchannel. In certain embodiments, the power source is operated by a user to deliver a user-defined voltage to the device.

In another embodiment, the system may also include a light source comprising a plurality of LEDs arranged around at least one microchannel. In another embodiment, the system may include a light source comprising a plurality of LEDs focused by lenses arranged around at least one microchannel. By using multiple LEDs, with or without lenses, more power can be supplied per unit volume of the at least one microchannel to increase the fluorescence intensity emitted by the quantum dots.

In another embodiment, the at least one microchannel is positioned such that an LED light source is provided at one end of the at least one microchannel. A mirror may be provided adjacent to at least a portion of the microchannel to concentrate the fluorescent emissions of the quantum dots to a CCD-based optical detection system. In another embodiment, a first LED can be provided at one end of the microchannel and a second LED can be provided at the opposite end of the microchannel, to enhance the excitation energy and thus the fluorescent emission of the quantum dots. For example, an LED light source can be provided adjacent to at least one microchannel to provide excitation energy to the quantum dots, and a CCD-based detection system measures fluorescent emissions from one end of the at least one microchannel.

### Binding of Quantum Dot Conjugates to Biomarkers

One method of measuring the concentration of a biomarker in a sample is to conjugate quantum dots to the biomarker and then to detect and quantify the presence of the quantum dots by fluorescence. The conjugation of quantum dots to a biomarker can be done by conjugating a quantum dot to an intermediary, such as a targeting moiety, which is selected based on its ability to specifically bind to a biomarker of interest.

A quantum dot conjugate comprises at least one quantum dot (i.e., a semiconductor nanocrystal) that can be detected by means of its fluorescent properties. Quantum dots are ultra-sensitive non-isotopic reporters of biomolecules in vitro and in vivo. Quantum dots are attractive fluorescent tags for biological molecules due to their large quantum yield and photostability. As such, quantum dots overcome many of the limitations inherent to the organic dyes used as conventional fluorophores. Quantum dots range from 2 nm to 10 nm in diameter, contain approximately 500-1000 atoms of materials such as cadmium and selenium, and fluoresce with a broad absorption spectrum and a narrow emission spectrum.

A water-soluble luminescent quantum dot, which comprises a core, a cap and a hydrophilic attachment group is well known in the art and commercially available (e.g. Quantum Dot Corp. Hayward, Calif.; Invitrogen, Carlsbad, Calif.; U.S. Pat. No. 7,192,785; U.S. Pat. No. 6,815,064). The core comprises a nanoparticle-sized semiconductor. While any core of the IIB VIB, IIIB VB or IVB-IVB semiconductors can be used, the core must be such that, upon combination with a cap, a luminescence results.

The cap or shell is a semiconductor that differs from the semiconductor of the core and binds to the core, thereby forming a surface layer on the core. The cap must be such that, upon combination with a given semiconductor core, a luminescence results. Two of the most widely used commercial quantum dots come with a core of CdSe or CdTe with a shell of ZnS and emissions ranging from 405 nm to 805 nm.

The attachment group, refers to any organic group that can be attached, such as by any stable physical or chemical association, to the surface of the cap of the quantum dot. In one embodiment, the attachment group can render the quantum dot water-soluble without rendering the quantum dot no longer luminescent. Accordingly, the attachment group comprises a hydrophilic moiety. In one aspect, the attachment group may be attached to the cap by covalent bonding and is attached to the cap in such a manner that the hydrophilic moiety is exposed. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. In another aspect, quantum dot may be rendered water soluble by capping the shell with a polymer layer that contains a hydrophobic segment facing inside towards the shell and a hydrophilic segment facing outside. The hydrophilic layer can be modified to include functional groups such as —COOH and —NH₂ groups for further conjugation to proteins and antibodies or oligonucleotides as described in Chan and Nie, 1998, (Science 281:2016-8), Igor et al., 2005, (Nature Materials 4:435-46), Alivisatos et al., 2005, (Annu. Rev. Biomed. Eng. 7:55-76) and Jaiswal et al., 2003, (Nature Biotech. 21:47-51) and incorporated herein in their entirety by reference.

A quantum dot can be conjugated to a targeting moiety. The targeting moiety specifically binds to the biomarker of interest and may comprise an antibody, a peptidomimetic, a polypeptide or aptamer, a nucleic acid or any other molecule provided it binds specifically to a biomarker of interest.

In another embodiment, the quantum dot may be conjugated to a targeting moiety comprising a nucleic acid binding moiety. The nucleic acid binding moiety may comprise any nucleic acid, protein, or peptide that binds to nucleic acids, such as a DNA binding protein. A preferred nucleic acid is a single-stranded oligonucleotide comprising a stem and loop structure and the hydrophilic attachment group is attached to one end of the single-stranded oligonucleotide.

The antibody or nucleic acid can be attached to the quantum dot, such as by any stable physical or chemical association, directly or indirectly by any suitable means. Quantum dot conjugation may be achieved by a variety of strategies that include but are not limited to passive adsorption, multivalent chelates or classic covalent bond formation described in Jaiswal et al., 2003 (Nature Biotechnol. 21:47-51) and incorporated by reference herein.

The covalent bond formation is the simplest in execution and hence widely used for conjugation. The antibody or nucleic acid is attached to the attachment group directly or indirectly through one or more covalent bonds. If the antibody is attached indirectly, the attachment preferably is by means of a “linker,” i.e., any suitable means that can be used to link the antibody or nucleic acid to the attachment group of the water-soluble quantum dot. The linker should not render the water-soluble quantum dot water-insoluble and should not adversely affect the luminescence of the quantum dot. Also, the linker should not adversely affect the function of the attached antibody or nucleic acid. Crosslinkers, e.g. intermediate crosslinkers, can be used to attach an antibody to the attachment group of the quantum dot. Ethyl-3-(dimethylaminopropyl) carbodiimide (EDAC) is an example of an intermediate crosslinker. Other examples of intermediate crosslinkers for use in the present invention are known in the art. See, e.g., Bioconjugate Techniques (Academic Press, New York, (1996)).

In one embodiment, amine groups on quantum dots are treated with a malemide group containing a crosslinker molecule. These “activated” quantum dots can be then be directly conjugated to a whole antibody molecule. However the direct conjugation may result in steric hindrance restricting access of the antibody to the antigen of interest. In those instances where a short linker could cause steric hindrance problems or otherwise affect the functioning of the targeting moiety, the length of the linker can be increased, e.g., by the addition of from about a 10 to about a 20 atom spacer, using procedures well-known in the art. One possible linker is activated polyethylene glycol, which is hydrophilic and is widely used in preparing labeled oligonucleotides.

The Stretptavidin Biotin reaction provides another conjugation method where the biotinylated protein/biomolecule is attached to a streptavidin coated quantum dot.

One of skill in the art will appreciate that it may be desirable to detect more than one antigen or protein of interest in a sample. Therefore, in particular embodiments, at least two antibodies directed to two distinct antigens or proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

Quantum dots are conjugated to antibody fragments using a heterobiofunctional crosslinker 4-(maleimidomethyl)-1-cyclohexanecarboxylic acid N-hydroxysuccinimide ester (SMCC). The commercial Quantum dots (Invitrogen Corporation, Carlsbad, Calif.) come with —NH₂ groups on their surface. These amino groups are reacted with the crosslinker SMCC to create malemide groups on the quantum dots surface. Antibodies of interest are reduced by DTT (Dithiothreitol) and disulfide bonds are broken to create thiol (—SH) groups. The final conjugation relied on the covalent bond formed between the malemide group on activated quantum dots and the thiol group on the antibodies. The ratio of antibody conjugated to quantum dots 1:4 and the typical yield of the reaction at the end of conjugation procedure is anywhere between 500 μl to 800 μl.

Table I presents a list of quantum dots conjugated to antibodies using the procedure outlined above:

TABLE 1
Different color QDs conjugated to various antibodies.

| | | Stock |
|---|---|---|
| Quantum Dots | Antibodies | Concentration |
| QD565 | MPO (Santa Cruz BT) | 1.2 μM |
| QD655 | MPO (Santa Cruz BT) | 500 nM |
| QD655 | Anti-Testosterone | 1.5 μM |
| QD605 | Anti-TNFα | 1 μM |
| QD705 | Anti-TNFα | 1.2 μM |
| QD 605 | Anti-IL-1α | 1.5 μM |
| QD 705 | Anti-IL-1α | 1.5 μM |

### Detection Using Quantum Dot as Fluorophores

Given the disclosure set forth herein, the skilled artisan will understand how to use any methods available in the art for identification or detection of an analyte, including without limitation, a protein, nucleic acid, or a biomolecule of interest. The present invention is suitable for detecting any type of chemical compound. In some embodiments, the methods of the present invention for detecting a molecule of interest comprise any method that determines the quantity or the presence of the biomarker protein or nucleic acid.

In one embodiment, the biomarker of interest is detected at the protein level. The method comprises contacting the sample with a quantum dot-antibody conjugate, wherein the antibody of the conjugate specifically binds to the biomarker protein, and detecting fluorescence, wherein the detection of fluorescence indicates that the conjugate bound to a protein in the sample.

In another embodiment, the target molecule of interest is detected at the nucleic acid level. The method comprises contacting the sample with a quantum dot-conjugate, wherein the targeting moiety of the conjugate specifically binds to the nucleic acid, and detecting residual fluorescence, wherein the detection of fluorescence indicates that the conjugate bound to the nucleic acid in the sample. Preferably, the targeting moiety of the conjugate is a nucleic acid. Alternatively, the targeting moiety of the conjugate is a protein or a fragment thereof that binds to a nucleic acid, such as a DNA binding protein.

The term “probe” refers to any molecule that is capable of selectively binding to a specifically intended target molecule, for example, a nucleotide transcript or a protein encoded by or corresponding to a target molecule. Probes can be synthesized by one of skill in the art, or derived from appropriate biological preparations. As contemplated in the present invention, a probe may be conjugated to a quantum dot of a particular size. Examples of molecules that can be used as probes include, but are not limited to, RNA, DNA, proteins, antibodies, and organic molecules.

The present invention also provides a method whereby two or more different target molecules and/or two or more regions on a given target molecule can be simultaneously detected in a sample. The method involves using a set of quantum dot conjugates, wherein each of the conjugates in the set has a differently sized quantum dot or a quantum dot of different composition attached to a targeting moiety that specifically binds to a different target molecule or a different region on a given target molecule in the sample. In an embodiment, the quantum dot of the conjugates range in size from 2 nm to 6.5 nm, which sizes allow the emission of luminescence in the range of blue to red. The quantum dot size that corresponds to a particular color emission is well-known in the art. Within this size range, any size variation of quantum dot can be used as long as the differently sized quantum dot can be excited at a single wavelength and differences in the luminescence between the differently sized quantum dot can be detected. In another embodiment, the differently sized quantum dot have a capping layer that has a narrow and symmetric emission peak. Similarly, quantum dot of different composition or configuration will vary with respect to particular color emission. Any variation of composition between quantum dot can be used as long as the quantum dot differing in composition can be excited at a single wavelength and differences in the luminescence between the quantum dot of different composition can be detected. Detection of the different biomarkers in the sample arises from the emission of multicolored luminescence generated by the quantum dot differing in composition or the differently sized quantum dot of which the set of conjugates is comprised. This method also enables different functional domains of one or more single proteins, for example, to be distinguished.

Accordingly, the present invention provides a method of simultaneously detecting two or more different biomarkers and/or two or more regions of a given biomarker in a sample. The method comprises contacting the sample with two or more conjugates of a water-soluble quantum dot and an antibody, wherein each of the two or more conjugates comprises a quantum dot of a different size or composition and an antibody that specifically binds to a different molecule or a different region of a given target molecule in the sample. The method further comprises detecting luminescence, wherein the detection of luminescence of a given color is indicative of a conjugate binding to a molecule in the sample.

### Diagnostic Assays

The present invention has application in various diagnostic assays for the detection of various diseases, disorders, conditions, and the like. That is, the present invention can be used to evaluate the presence or absence of any known or unknown compound present in a body sample. For example, in certain embodiments, the device and method described herein is used to detect the presence or absence of a biomarker. The present invention can be used to detect a biomarker by removing a sample to be tested from a patient; contacting the sample with a water-soluble quantum dot conjugated to a targeting moiety that specifically binds to a biomarker, and detecting the luminescence, wherein the detection of luminescence indicates the presence of the desired biomarker. In these cases, the sample can be a cell or tissue biopsy or a bodily fluid, such as blood, serum, urine, or fecal sample.

The biomarker can be a protein, a nucleic acid or enzyme associated with a given disease, the detection of which indicates the existence of a given disease state. The detection of a disease state can be either quantitative, as in the detection of an over- or under-production of a protein, or qualitative, as in the detection of a non-wild-type (mutated or truncated) form of the protein. In regard to quantitative measurements, preferably the luminescence of the quantum dot conjugate is compared to a suitable set of standards. A suitable set of standards comprises, for example, the quantum dot conjugate of the present invention in contact with various, predetermined concentrations of the biomarker being detected. One of ordinary skill in the art will appreciate that an estimate of, for example, amount of protein in a sample, can be determined by comparison of the luminescence of the sample and the luminescence of the appropriate standards, as described in detail elsewhere herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Microfabricated QLISA Biosensors with an Embedded Mixing Element

The experiments presented herein demonstrate the superior mixing and sensitivity of a QLISA microfluidic biosensor with integrated electrodes to facilitate enhanced electrothermal flow of the sample constituents resulting from joule heating.

The developed microfluidic immunoassay platform presented herein is based upon a predicate, poly(methyl methacrylate) (PMMA) microchannel immunoassay device coupled to an optical excitation and detection system for analyte detection. The heterogeneous immunoassay utilizes a sandwich immunoassay format with quantum dot conjugated detection antibodies to generate an optical signal and subsequently calculate analyte concentration. The presently described platform comprises fabricated microchannels on a PMMA substrate hermetically sealed onto a silicon substrate patterned with electrodes for parallel sample analysis and electrothermal mixing to enhance reaction kinetics and decrease incubation time. The studies presented herein demonstrate comparable limits of detection and sensitivity to standard microtiter based immunoassays while requiring less than 1 μl of sample and significantly reduced reagent consumption over conventional immunoassay methods. The platform described herein exhibits superior mixing and assay sensitivity, effectively reducing sample incubation times and reagent consumption compared to the standard microtiter plate immunoassay format.

The materials and methods employed in these experiments are now described.

Biosensor Fabrication

Two biosensor prototypes were microfabricated, one with integrated electrodes and one without electrodes in order to assess the effect of electrothermal mixing upon immunoassay kinetics. Both designs used a micropatterned piece of PMMA containing the microchannel design, which was then sealed to either a silicon substrate patterned with electrodes or another piece of PMMA. An overview of the fabrication process for both the micropatterned PMMA piece (FIG. 1A) and the integrated electrode design (FIG. 1B) are depicted in FIG. 1. The micropatterned PMMA piece containing the microchannels was fabricated by first patterning SU-8 2150 onto a 2.5″ square piece of mirror finished copper (McMaster Carr). SU-8 2150 was spin coated onto the mirrored side of the copper substrate to a final thickness of 250 μm and patterned using standard techniques of photolithography. The SU-8 patterned copper plate was then electroplated under stirring with an applied current of 0.2 A for 90 minutes using an electroplating bath. The SU-8 was then removed from the copper substrate using a novel thermal delamination technique taking advantage of the differences in the coefficients of thermal expansion between the copper and SU-8. The copper master was then used to transfer the microchannel design onto PMMA using a Carver hydraulic thermal press set to 120° C. under an applied force of 1000 lb for 5 minutes. The final dimensions of the microchannels on the PMMA were 220 μm in width and 110 μm high with a length of 3 cm. The micropatterned piece of PMMA was then sealed onto another piece of PMMA using solvent assisted thermal bonding (95% ethanol water mixture) and using the Carver thermal press (1000 lb of force at 75° C. for 5 minutes).

Alternatively, the micropatterned PMMA piece was hermetically sealed onto a silicon substrate patterned with electrodes using a UV curable resin. Single channel pieces were cut from the imprinted pieces above. Then the microchannel was vertically aligned with fabricated electrodes, and a clamp was used to hold the assembly together. A UV-curable adhesive resin was introduced between the contact area of PMMA channels and silicon substrate. The resin filled up the gap due to capillary action but the microchannel was kept open (the resin did not continue flowing into the microchannel) due to the capillary pressure drop. 3 min UV exposure was applied to complete the bonding process.

ETF Simulation

Static heterogeneous immunoassays may be modeled as a diffusion reaction system and have been well defined within the literature. In certain embodiments, the present device utilizes integrated electrodes to produce a thermal gradient within the microchannel, inducing electrothermal fluid mixing. The model used to calculate the theoretical particle velocity under electrothermal flow (ETF) is based upon previous models within the literature. Briefly, the ETF effect may be simulated using COMSOL by simultaneously solving for the electric potential of the system (1), the Navier-Stokes equation that includes the electrothermal force term (2) and the convection-conduction equation (3).

$$\begin{matrix}∇ ( ɛ ∇ V ) = 0 & ( 1 ) \\ ρ \frac{D \vec{V}}{Dt} = - ∇ p + η {∇}^{2} \vec{V} + {\vec{f}}_{ETF} & ( 2 ) \\ k {∇}^{2} T + σ {E}^{2} = ρ {c}_{p} \vec{V \cdot ∇ T} & ( 3 )\end{matrix}$$

Immunoassay

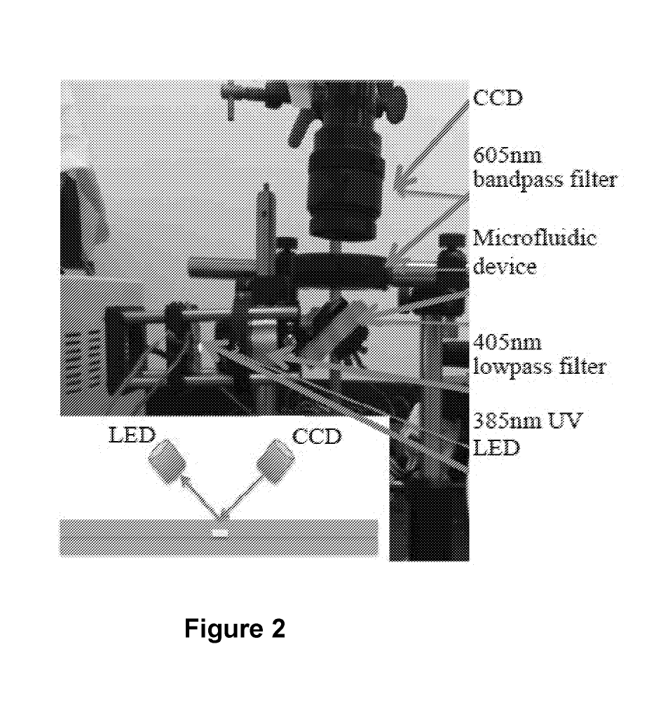

An immunoassay for myeloperoxidase (MPO) was performed. Briefly, capture antibody was covalently immobilized onto the lumen of the PMMA microchannel using EDC and sulfo-NHS. Antigen was then introduced into the microchannel and allowed to react for 60 minutes followed by several washes using 0.05% Tween 20 (1×PBS pH 7.4). Quantum dot conjugated capture antibody is then incubated for 60 minutes and allowed to react followed by several washes using 0.05% Tween 20 (1×PBS pH 7.4). The device is then imaged using the optical setup shown in FIG. 2.

The results of the experiments are now described.

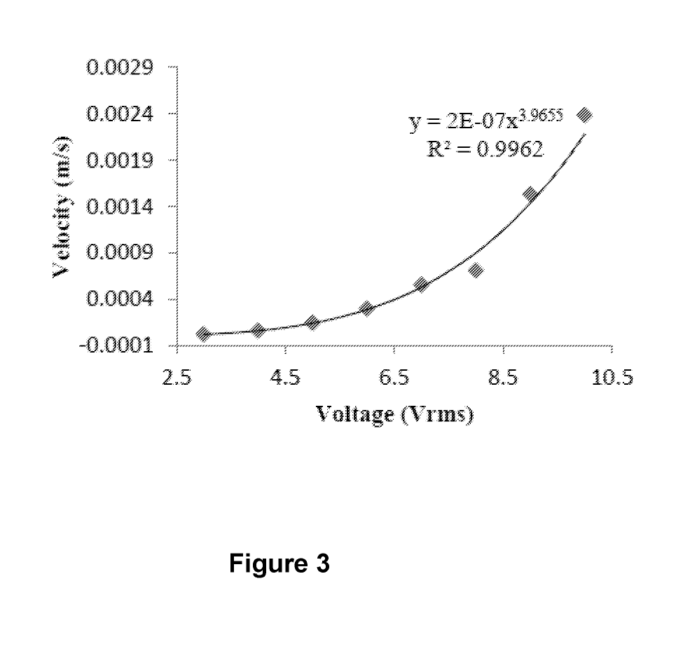

The particle movement due to electrothermal flow (ETF) was observed using 2 μm polystyrene microbeads in 0.1M PBS buffer solution. The particle movement due to ETF was observed as the amplitude and frequency of the applied voltage was varied. Below 3 Vrms no particle motion was observed. As the voltage was increased, particles became more and more active until the electrodes were damaged at 10 Vrms. The experimental results had a good agreement with the numerical simulation shown in FIG. 3. A set of parameters (6 Vrms, 200 kHz) was chosen for immunoassay experiments.

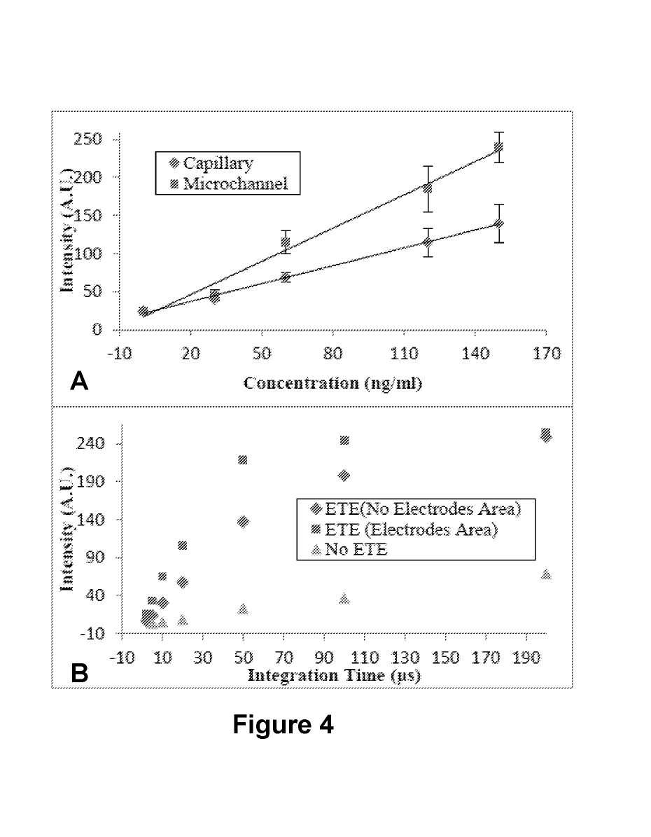

Immunoassay performance between a predicate capillary based immunosensor and the developed microfluidic immunosensor platform was compared (FIG. 4) in the absence of ETF mixing. As evident from FIG. 4A, it is shown that the rectangular microchannel resulted in an increased sensitivity as well as a smaller standard deviation of measurement. A similar microfluidic assay was developed based upon previous work for myeloperoxidase (MPO), which was the basis for subsequent ETF enhancement experiments. Electric field was applied during MPO and quantum dot-mAb incubation steps. FIG. 4B shows significant improvement with ETF mixing. Without ETF mixing, the fluorescent signal was very low, indicating that there is limited binding between pAb and MPO, and between MPO and quantum dot-mAb within 5 minutes. However, with ETF mixing, the binding efficiency was greatly improved. Signal intensity reaches the saturation at 50 μs of shutter speed.

Two types of quantum dots based PMMA microchannel immunosensor devices have been developed in this study. The high yield of quantum dots enables the devices the capability of detecting nanomolar quantities of analytes. With the enhancement of electrothermal effect, the reaction and response time is significantly shortened. It reduced the time need for the whole assay by nearly 50%, while the signal intensity was comparable to normal assay.

### REFERENCES

Babu et al., 2009, Biosensors & Bioelectronics, 24(12): 3467-3474.
Gervais and Jensen, 2006, Chemical Engineering Science, 61(4): 1102-1121.
Hu et al., 2007, Biosensors & Bioelectronics, 22(7): 1403-1409.
Squires et al., 2008, Nature Biotechnology, 26(4): 417-426.
Zimmermann et al., 2005, Biomedical Microdevices, 7(2): 99-110.
Sigurdson et al., 2005, Lab on a Chip, 5(12): 1366-1373.
Hart et al., 2008, J Vis Exp, (17): 813.
Oh et al., 2009, Lab on a Chip, 9(1): 62-78.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting an analyte in a sample, the method comprising:
providing a sample to at least one microchannel coated with an antibody, the sample potentially including an analyte;
contacting the sample with a conjugate comprising a quantum dot and an antibody that specifically binds to the analyte;
increasing electrothermal flow of the sample;
energizing the quantum dot with a light source;
detecting fluorescent emission from the quantum dot; and
correlating the fluorescent emission to the presence of or the concentration of the analyte in the sample.

2. The method of claim 1, wherein the electrothermal flow is increased by DC electroosmotic transverse mixing.

3. The method of claim 1, wherein the analyte is selected from the group consisting of an enzyme, an adhesion molecule, a cytokine, a protein, a lipid mediator, an immune response mediator, and a growth factor.

4. The method of claim 1, wherein the electrothermal flow is increased by applying a electricity to an electrode within at least a portion of the at least one microchannel.

5. The method of claim 4, wherein the electricity has a voltage between about 1 V_{RMS} and about 10 V_{RMS}.

6. The method of claim 4, wherein the electricity has a frequency of about 0.1 Hz to about 100 MHz.

7. The method of claim 4, wherein the electricity has a voltage of about 6 V_{RMS} and a frequency of about 200 kHz.

8. The method of claim 1, wherein the volume of the sample is about 0.1×10⁻¹⁰ m³ to about 0.1×10⁻⁵ m³

9. The method of claim 1, wherein the volume of the sample is less than or equal to about 1 μL.

10. The method of claim 1, wherein detection of the analyte occurs in about 1 minute to about 60 minutes.
